(19) United States
(12) Patent Application Publication
(10) Pub. No.: US 2006/0239931 A1
(43) Pub. Date: Oct. 26, 2006

(54) Pharmaceutical aerosol composition

(76) Inventors: James Edward Eyles, Wiltshire (GB); Gary John Phillips, Wiltshire (GB); Michael Patrick Maidment, Wiltshire (GB); Ethel Diane Williamson, Wiltshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/542,449

(22) PCT Filed: Jan. 14, 2004
(86) PCT No.: PCT/GB04/00104

§ 371 Date: Dec. 13, 2005

(30) Foreign Application Priority Data
Jan. 15, 2003 (GB) 0300885.1

(51) Int. Cl. A61K 39/08; A61K 39/07; A61K 39/02; A61K 9/14; A61L 9/04

(52) U.S. Cl. 424/46; 424/239.1; 424/246.1; 424/234.1; 424/490

(57) ABSTRACT

An aerosol formulation comprising a biodegradable microsphere comprising a non-living reagent, such as a sub-unit vaccine, that produces a protective immune response in a mammal to whom it is administered. Nebulizers and inhalers containing such formulations are also described and claimed.

The present invention relates to pharmaceutical compositions, and in particular to compositions comprising immunogens, used in the prophylactic and therapeutic treatment of infections.

The option to self administer vaccines by inhalation, for example using a nebulizer or inhaler such as a dry powder inhaler, would be advantageous from a logistical standpoint and may be particularly effective for protecting individuals from pathogens that affect or utilise the respiratory tract as a portal of entry into the body.

U.S. Pat. No. 6,428,771 describes a method for controlled drug delivery to the pulmonary system using microparticles incorporating the drug. Particles are described as having a diameter of from 0.5 and 10 μm. It is suggested that the drug may in fact comprise an antigen intended to elicit an a protective immune response. However this is not demonstrated.

Furthermore, administration of in particular non-living vaccines, such as sub-unit vaccines, has not yet been found to give effective protection using this mode of administration (see for example C. W. Purdy et al., Current Microbiology, (1998), 37, p5).

The applicants have found that biodegradable microspheres, containing antigen, can engender immunological responses following delivery to experimental animals in the form of an aerosol, provided the microspheres are of a type which are delivered most efficiently to the lung.

According to the present invention there is provided an aerosol formulation comprising a biodegradable microsphere of average diameter of from 0.5 to 5 μm and comprising a non-living reagent that produces a protective immune response in a host mammal to whom it is administered.

As used herein, the term “non-living reagent” refers to immunogens such as polypeptides or proteins, which are derived for example from a pathogen such as a bacteria, virus or fungi. It also refers to inactivated microorganisms such as heat or chemically killed bacteria and/or viruses.

The term “aerosol” refers to a formulation that is deliverable in the form of a dispersion of a solid and/or liquid in a gas. These may be prepared from suspensions of the formulation in a liquid such as water, using a device such as a nebulizer, or from dry powders using a dry powder inhaler. In the case of the nebulized aerosol, the dispersion comprises essentially wet microspheres in air.

The term “average diameter” as used herein, refers to the mean mass aerodynamic diameter of the microspheres. Mean mass aerodynamic diameter is a measurement of particle size in an aerosol, which is the most relevant measurement when trying to predict if particles are respirable.

These formulations are effective in the administration of reagents, which are capable of generating a protective immune response in an animal, particularly a mammal, to which it is administered. Examples of such agents include antigenic polypeptides as well as nucleic acid sequences which may encode these polypeptides and which are known as “DNA” vaccines.

Suitable polypeptides are sub-unit vaccines and others, such as diptheria toxoid, tetanus toxoid, Botulinun toxin FHc and Bacillus anthracis protective antigen (PA).

As used herein the expression “polypeptide” encompasses proteins or epitopic fragments thereof.

Suitable polypeptides are sub-unit vaccines.

In a preferred embodiment, the formulation of the invention comprises a biologically active agent which is capable of generating a protective immune response against Yersinia pestis. The agent is suitably a sub-unit vaccine, for example V antigen of Y. pestis or an immunologically active fragment thereof or a variant of these, or the F1 antigen of Y. pestis or an immunologically active fragment thereof or a variant of these, or a combination of these. In particular as described in WO 96/28551, preferred vaccine comprises a combination of the F1 and V antigens.

As used herein, the term “fragment” refers to a portion of the basic sequence that includes at least one antigenic determinant. These may be deletion mutants. One or more epitopic region of the sequence may be joined together.

The expression “variant” refers to sequences of nucleic acids that differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 75% identical, and more preferably at least 90% identical to the base sequence. Identity in this case can be determined using available algorithms such as the widely used BLAST program.

The applicants have found that nebulization of PLA microspheres generates a respirable ‘plume’ of aerosolised particles, and this approach can be used to deliver immunogens to the respiratory tracts of experimental animals. Similar plumes could be produced using other forms of inhaler such as dry powder inhalers.

Microspheres used are suitably small enough to allow them to be administered to the deep lung using a conventional nebulizer or inhaler. For this purpose, microspheres will be less that 5 μm average diameter, preferably less than 3 μm average diameter, for instance from 0.5-3 μm, or more preferably from 1-3 μm and most preferably with an average diameter of between 1 and 1.5 μm.

Suitably 0% of microspheres have an aerodynamic diameter above 10 μm. More suitably, 0% of microspheres have an aerodynamic diameter above 9 μm, and preferably 0% of microspheres have an aerodynamic diameter above 6 μm.

Suitably, at least 90%, and preferably at least 95% of the microspheres in the formulation have an aerodynamic diameter of less than 5 μm, preferably with at least 80% of particles having a mean mass aerodynamic diameter of less than 3 μm.

By using microspheres of this size, efficient delivery of reagent into the deep lung is achieved. This is important in the delivery of reagents of this type as it is essential to achieve the highest concentrations of reagent, which can feasibly and safely be delivered in order to achieve the protective immune response.

Microspheres are suitably biodegradable and are produced from polymeric material. The polymeric material is suitably a biogdegradable polymer other than a lipid, and in particular a biodegradable polyester. A particularly suitable polymer for use in the preparation of microcapsules is Poly-lactide (PL) although other polymers such as poly(lactide-co-glycolide) PLGA may also be employed.

The microspheres may optionally further comprise agents which stabilise emulsions such as polyvinylalcohol (PVA), dipalmitoylphophatidylcholine (DPPC), or methyl cellulose, and preferably polyvinylalcohol.

Suitably the non-living reagent is encapsulated within the microspheres (microcapsules). This again ensures the a high dose of the reagent is delivered to the lung which is important if a protective immune response is to be generated.

Microcapsules are suitably prepared using conventional methods such as the double emulsion/solvent evaporation method, as described for example by Beck et al., 1979, Fertility and Sterility, 31:545-551.

The encapsulation is suitably achieved using a double emulsion solvent evaporation method, in which a first emulsion is formed with the non-living reagent, and the structural polymer, mixing this with an aqueous phase (suitably without structural polymer) to form a secondary emulsion, evaporating solvent and isolating small microspheres. In particular, the pharmaceutically active ingredient is dissolved or suspended in an aqueous solution, which optionally includes an emulsifier such as PVA. The emulsifier, where present is suitably included at low concentrations for example of less than 5% w/v. This solution or suspension in then mixed with a solution of the high molecular weight structural polymer in an organic solvent such as dichloromethane. A primary emulsion is then formed, in particular by sonication of the mixture. The primary emulsion in then added to a secondary aqueous phase, which preferably includes an emulsifier with vigorous stirring. Solvent is then preferably evaporated, conveniently at room temperature.

Microspheres can then be recovered, for example by centrifugation followed by lyophilisation.

The formulations of the invention may comprise microspheres per se which are optionally preserved, for example by lyophilisation, or the microspheres may be combined with a pharmaceutically acceptable carrier or excipient. Examples of suitable carriers include solid carriers as is understood in the art for use in nebulizers.

In a particularly preferred embodiment, the formulation further comprises the non-living reagent in free form. The ratio of the amounts of the free reagent to the reagent associated with the microspheres used in the composition may vary depending upon the particular agents being employed. Suitably the ratio of the free reagent to the reagent contained in the microspheres is in the range of from 1:20 to 2:1 and preferably at about 1:10.

The formulation of the invention may further comprise an adjuvant in order to enhance the immune response to the biologically active material administered. Suitable adjuvants include pharmaceutically acceptable adjuvants such as Freund's incomplete adjuvant, alhydrogel, aluminium compounds and, preferably adjuvants which are known to up-regulate mucosal responses such as CTB, the non-toxic pentameric B subunit of cholera toxin (CT) or mutant heat-labile toxin (mLT) of E. coli. They may also include immunomodulators such as cytokines and CpG motifs.

Other adjuvant types are described in International Patent Application Nos. WO00/56282, WO00/56362 and WO00/56361.

Suitably the formulations are in unit dosage form. This will vary depending upon the nature of the active agent being employed, the nature of the patient, the condition being treated and other clinical factors. In general however, the formulations of the invention will comprise approximately 0.5 to 10 w/w of non-living reagent.

Exposed animals, in this case, mice, respond with a humoral response. It has also been found that experimental animals can be protected by this treatment from a lethal challenge with a pathogen such as the plague causing bacteria (Yersinia. pestis) by exposure to aerosolised microspheres containing recombinant V antigen. The applicants are therefore the first to demonstrate the successful aerogenic immunisation using non-living vaccines.

Dosages of the formulations of the invention will depend upon various factors such as the the nature of the patient, the antigen used etc. and will be determined according to known clinical practice.

It has been found that in a particularly preferred embodiment, each administration of microsphere preparation to a mouse contains from 1-100 μg, suitably from 30-50 μg and most preferably about 40 μg of each of said antigens. Preferably the dosage to humans and mammals would be of the same order in terms of mg/Kg.

According to a further aspect of the invention, there is provided a nebulizer or inhaler comprising a formulation as described above.

Dry powder inhalers may be particularly useful in the context of the invention as dry vaccine formulations, which would be used therein, are stable at ambient temperatures.

In yet a further aspect, the invention provides the use of microspheres comprising a non-living reagent that produces a protective immune response in a mammal to whom it is administered, in the preparation of a vaccine for administration as an aerosol.

Further according to the invention there is provided a method of producing a protective immune response in a mammal in need thereof, said method comprising administering to the lung of said mammal, a protective amount of an aerosol formulation as described above.

The invention will now be particularly described by way of example with reference to the accompanying diagrammatic drawings in which:

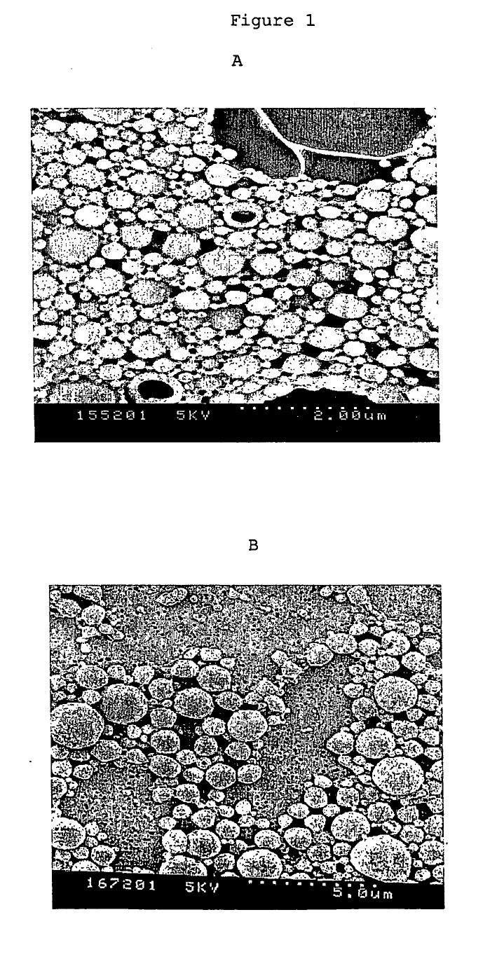

FIG. 1 is a micrograph showing the morphology of microspheres prior to (A) and after (B) nebulization;

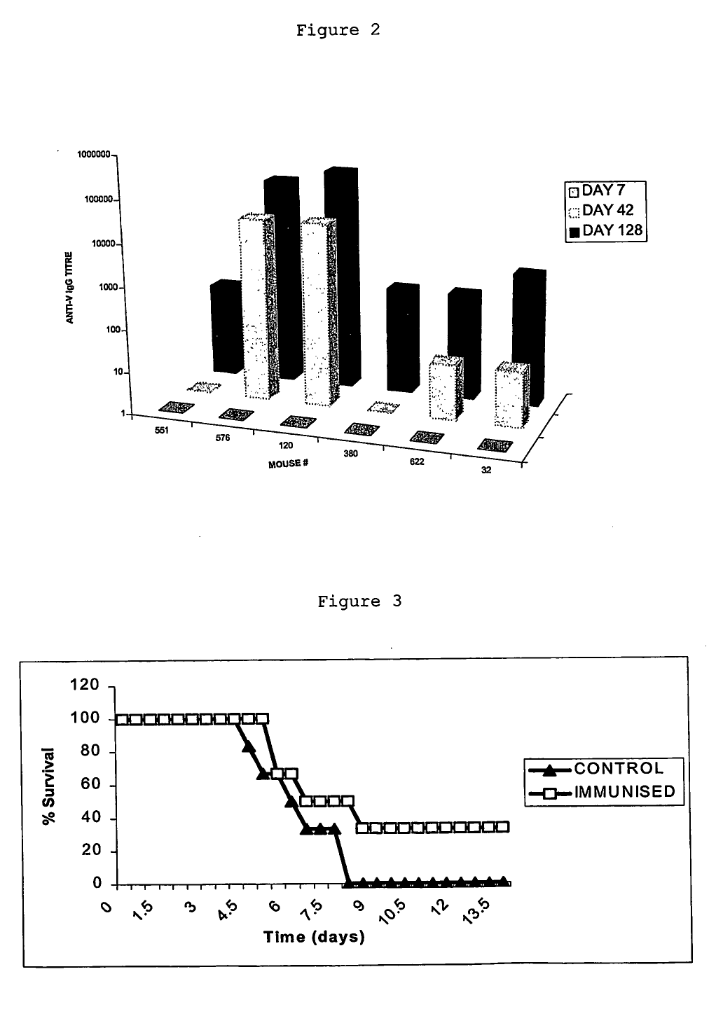

FIG. 2 is a graph showing serum anti-V IgG endpoint titre in 6 BALB/c mice exposed to aerosolised microspheres containing recombinant Yersinia pestis V antigen; and

FIG. 3 illustrates the survival of mice, previously exposed to aerosolised microspheres containing rV antigen, after subcutaneous injection of 6.5 MLDs Y. pestis;

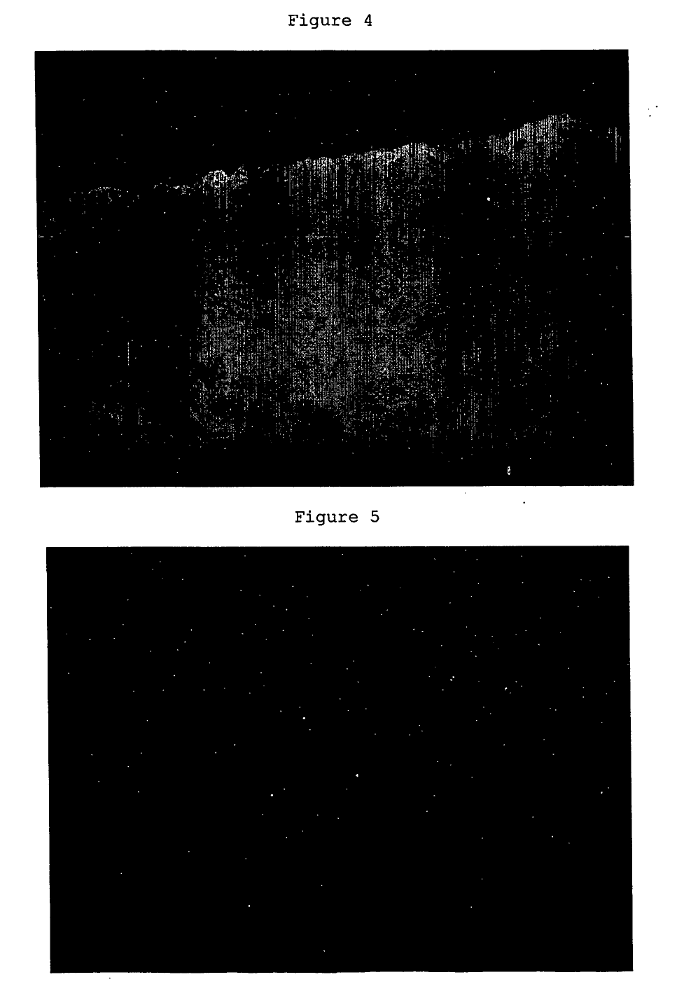

FIG. 4 is a fluorescence micrograph of lung taken 24 hours following exposure of mice to aerosolised microspheres loaded with FITC-BSA; and

FIG. 5 is a fluorescence micrograph of lung lymph node taken 24 hours following exposure of mice to aerosolised microspheres loaded with FITC-BSA.

### EXAMPLE 1

Poly-lactide (resomer L210) microspheres containing either BSA or recombinant V antigen from Y. pestis were fabricated using a modified double-emulsion solvent evaporation process. PLA, sold under the trade name Resomer L210, is a linear crystalline homopolymer with an inherent viscosity of approximately 3.6.

The polymer was used at a concentration of 1.38% w/v in dichloromethane (10 ml). An aqueous solution (0.5 ml) containing the antigen of interest (about 4 mg) was then added and the mixture stirred at high speed to generate an emulsion. This emulsion was then added to a second aqueous phase and mixed together at high speed.

The solvent was then evaporated to leave an aqueous suspension of antigen-loaded microspheres.

Particles were aerosolised using a Sidestream® nebulizer. An aerosol particle sizer was used to analyse size characteristics. Samples were collected using a three stage liquid impinger and analysed using scanning electron microscopy, SDS PAGE and western blotting procedures.

6 female BALB/c mice were exposed to a stream of aerosolised microspheres in a head only exposure line. 77 mg of rV loaded microspheres were suspended in 17 ml of free V (at 0.4 mg ml⁻¹ in distilled water). Mice were exposed to the aerosolised microspheres for three ten minute runs, during which time approximately 3 ml of particle suspension was nebulized each run. The was repeated on days 0, 21 and 107 of the experiment and sera analysed for the presence of anti-V IgG using an indirect ELISA. In order to assess the extent of protection afforded by inhalation of the V loaded microspheres, mice were injected subcutaneously with 6.3MLDs Y. pestis (GB strain) on day 136 of the experiment.

Results and Discussion

Microspheres had a loading of 3.8% w/w (BSA) and 3.3% w/w (rV). Following aerosolisation the BSA loaded particles had a mass median aerodynamic diameter of 1.3±1.4 μm, with 93% of the particles under 3 μm. Following nebulization, particles retained their morphology/topography (FIG. 1) and contained antigenic material as detected by Western Blotting.

Although there was some inter-animal variation in the serum antibody response to aerosolised Y. pestis rV antigen, all 6 mice seroconverted after three immunising doses (FIG. 2). Two of the six mice responded with antibody titres that were of significant magnitude to confer protection from injected challenge with plague causing bacteria (FIG. 3).

### EXAMPLE 2

Delivery of Microencapsulated Antigen to the Lung and Lung Lymph Node by Aerosolisation

Poly-lactide (resomer L210, Alfa chemicals UK) microspheres, containing FITC-BSA were fabricated using a modified double-emulsion solvent evaporation process. Particles were aerosolised using a Sidestream® nebulizer (Profile, UK). Female BALB/c mice were exposed to the aerosolised microspheres in a head only exposure chamber. 24 hours following exposure mice were killed and their lungs and lung's lymph nodes were extracted. Frozen sections were obtained from the extracted tissues using a cryostat. Frozen sections were examined for the presence of FITC-BSA loaded microspheres using a fluorescence microscope and the results for the lung and lung lymph nodes are shown in FIGS. 4 and 5 respectively.

The visualisation of punctate fluorescent material in the sections indicated the presnce of FITC-BSA loaded microspheres in the lung and lymph nodes. These data support the tenet that microspheres can reach enter the lower respiratory tract following nebulization. Furthermore, these data indicate that microspheres may be translocated from the lungs to the draining lymph nodes, following nebulization.

1. An aerosol formulation comprising a biodegradable microsphere of average diameter of from 0.5 to 5 μm comprising a non-living reagent that produces a protective immune response in a mammal to whom it is administered.

2. The formulation of claim 1 wherein the said non-living reagent is antigenic polypeptide or a nucleic acid sequences which may encode such a polypeptide.

3. The formulation of claim 2 wherein the said non-living reagent is a sub-unit vaccine.

4. The formulation of claim 1 wherein the said non-living reagent is diptheria toxoid, tetanus toxoid, Botulinum toxin FHc, Bacillus anthracis protective antigen (PA) or a polypeptide which is capable of generating a protective immune response against Yersinia pestis.

5. The formulation of claim 4 wherein the non-living reagent is the V antigen of Y. pestis or an immunologically active fragment thereof or a variant of these, or the F1 antigen of Y. pestis or an immunologically active fragment thereof or a variant of these, or a combination of these.

6. The formulation of claim 1 wherein the microspheres have an average diameter of less than 3 μm.

7. The formulation of claim 6 wherein the microcapsules have an average diameter of between 1 and 1.5 μm.

8. The formulation of claim 1 wherein the microspheres comprise a biodegradable polyester.

9. The formulation of claim 8 wherein the polyester comprises Poly-lactide (PL).

10. The formulation of claim 1 wherein the microcapsules are lyophilised.

11. The formulation of claim 1 wherein the non-living reagent is encapsulated within the microspheres.

12. The formulation of claim 1 which further comprises the non-living reagent in free form.

13. The formulation of claim 12 wherein the ratio of the amounts of the free reagent to the reagent associated with the microspheres is in the range of from 1:20 to 2:1.

14. The formulation of claim 1 in unit dosage form.

15. A nebulizer or inhaler comprising a formulation of claim 1.

16. (canceled)

17. A method of producing a protective immune response in a mammal in need thereof, said method comprising administering to the lung of said mammal, a protective amount of an aerosol formulation of claim 1.
